(19) United States
(12) Patent Application Publication
(10) Pub. No.: US 2022/0142736 A1
(43) Pub. Date: May 12, 2022

(54) DENTAL TREATMENT ROBOT

(71) Applicant: Hyung Joo KIM, Anseong-si (KR)

(72) Inventor: Hyung Joo KIM, Anseong-si (KR)

(21) Appl. No.: 17/582,427

(22) Filed: Jan. 24, 2022

Related U.S. Application Data
(63) Continuation of application No. PCT/KR2020/009884, filed on Jul. 27, 2020.

(30) Foreign Application Priority Data
Jul. 25, 2019 (KR) 10-2019-0090231

(51) Int. Cl. A61C 5/50; A61B 34/30; A61B 90/00; A61B 90/57

(52) U.S. Cl. CPC A61C 5/50; A61B 90/57; A61B 90/361; A61B 34/30

(57) ABSTRACT

A dental treatment robot is provided. The robot comprises: a head fixing unit for fixing a head of a patient; a jaw fixing unit for fixing a jaw of the patient in an opened state; a tooth fixing unit for fixing a treatment target tooth or a tooth around the treatment target tooth; a robot arm capable of moving in directions of at least three axes; a handpiece that is detachably attached to the robot arm and includes a rotating part for tooth preparation; and a base whose movement is fixed. The head fixing unit, the jaw fixing unit, and the tooth fixing unit are connected and fixed to the base, and the robot arm moves on the basis of the bae to carry out the tooth preparation.

### CROSS-REFERENCE TO RELATED APPLICATION(S)

This application is a continuation application of Patent Cooperation Treaty (PCT) International Application No. PCT/KR2020/009884 filed on Jul. 27, 2020, which claims priority to Korean Patent Application No. 10-2019-0090231 filed on Jul. 25, 2019. The entire contents of PCT International Application No. PCT/KR2020/009884 and Korean Patent Application No. 10-2019-0090231 are hereby incorporated by reference.

### FIELD OF THE INVENTION

The present invention relates to a dental treatment robot.

### BACKGROUND

Technology related to the fourth industrial revolution is rapidly affecting our society and real life. The dental community is also rapidly adapting to these technological and social movements. For example, data for impression taking are acquired through a three-dimensional scan without an impression material, and dentists and dental technicians can collaborate through information and communication technology wherever they are in the world.

However, traditional methods are maintained in the case of tooth preparation, which is the most frequently performed treatment in dentistry, and technology related to the fourth industrial revolution (e.g., robot technology) has not been actively introduced to the tooth preparation. The tooth preparation refers to a treatment for cutting off and removing a decayed part of a tooth.

Teeth are in less visible positions in a mouth, and a dentist who removes tooth decay should bend his/her back and neck while handling dental instruments including rapidly rotating parts. This causes an excessive physical load to the dentist, and the probability of developing back and neck disc disease or the like as an occupational disease is very high. This acts as a factor that lowers the quality of patient treatment. Further, since the tooth preparation is a sensitive procedure performed in an uncomfortable position, there is a big difference in the quality of the tooth preparation procedure between a novice dentist and a dentist with five or more years of experience. Carrying out the tooth preparation as desired is like an artist drawing a picture beautifully, and it takes time to achieve skilled techniques.

By performing a tooth preparation procedure using a robot, the difference in the quality of the tooth preparation procedure depending on the dentists may be overcome, the probability of medical accidents (e.g., injuring a patient's lip, cheek, tongue, or the like by various dental treatment instruments) may be reduced, and the number of personnel necessary for performing the procedure (e.g., dental hygienists for assisting the dentists) may be reduced.

### SUMMARY OF THE INVENTION

One object of the present invention is to provide a dental treatment robot, more specifically a dental treatment robot capable of carrying out tooth preparation and filling a cavity caused by the tooth preparation with a filling material such as a resin.

The representative configuration of the invention to achieve the above object is described below.

A dental treatment robot according to one embodiment of the invention comprises: a head fixing unit configured to fix a head of a patient; a jaw fixing unit configured to fix a jaw of the patient in an opened state; a tooth fixing unit configured to fix a treatment target tooth or a tooth around the treatment target tooth; a robot arm capable of moving in directions of at least three axes; a handpiece that is detachably attached to the robot arm and includes a rotating part for tooth preparation; and a base whose movement is fixed. The head fixing unit, the jaw fixing unit, and the tooth fixing unit are connected and fixed to the base, and the robot arm moves on the basis of the base to carry out the tooth preparation.

The robot arm may repeatedly move in a straight line defined by two inputted endpoints to remove tooth decay. Here, the robot arm repeatedly moves in the straight line in a state in which a direction of a longitudinal axis of the tooth is aligned parallel to a z-axis. The robot arm may move stepwise in the z-axis direction to remove deep-seated tooth decay.

In addition, there may be further provided other configurations according to the technical idea of the invention.

According to the invention, it is possible to provide a dental treatment robot, more specifically a dental treatment robot capable of carrying out tooth preparation and filling a cavity caused by the tooth preparation with a filling material such as a resin.

### BRIEF DESCRIPTION OF THE DRAWINGS

FIG. 1 is a table summarizing differences between a common surgical robot and a dental treatment robot according to the invention.

FIG. 2 illustrates a procedure of fixing a patient's body when tooth preparation is carried out with a dental treatment robot according to one embodiment of the invention.

FIG. 3 illustrates a situation of performing a tooth preparation procedure with a dental treatment robot in a state in which a patient's body, head, jaw, and tooth are fixed.

FIG. 4 specifically illustrates a dental treatment robot and a tooth fixing unit according to one embodiment of the invention.

FIG. 5 illustrates a handpiece to which a camera is attached.

FIG. 6 illustrates that a wiper is installed on a rotating type camera lens cover of a handpiece according to one embodiment of the invention.

FIGS. 7A, 7B, and 7C illustrate a situation of performing a tooth preparation procedure with a dental treatment robot according to one embodiment of the invention.

### DETAILED DESCRIPTION

In the following detailed description of the present invention, references are made to the accompanying drawings that show, by way of illustration, specific embodiments in which the invention may be practiced. These embodiments are described in sufficient detail to enable those skilled in the art to practice the invention. It is to be understood that the various embodiments of the invention, although different from each other, are not necessarily mutually exclusive. For example, specific shapes, structures, and characteristics described herein may be implemented as modified from one embodiment to another without departing from the spirit and scope of the invention. Furthermore, it shall be understood that the positions or arrangements of individual elements within each of the embodiments may also be modified without departing from the spirit and scope of the invention. Therefore, the following detailed description is not to be taken in a limiting sense, and the scope of the invention is to be taken as encompassing the scope of the appended claims and all equivalents thereof.

Hereinafter, various preferred embodiments of the present invention will be described in detail with reference to the accompanying drawings to enable those skilled in the art to easily implement the invention.

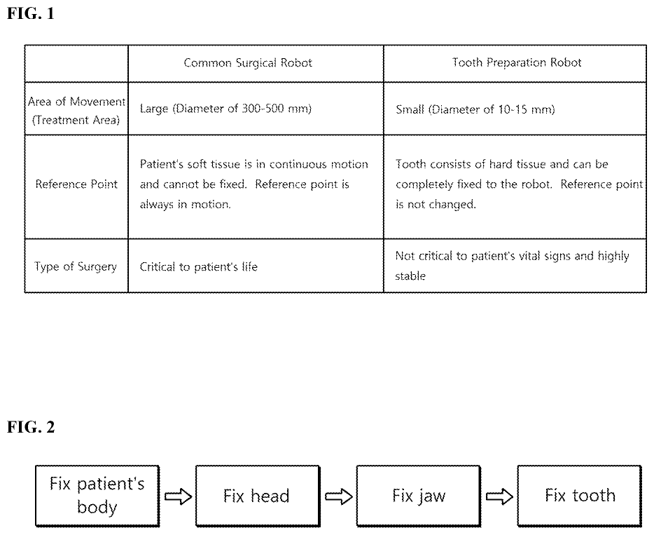

FIG. 1 is a table summarizing differences between a common surgical robot and a dental treatment robot according to the invention. FIG. 1 shows general differences from a common surgical robot available in the market, which are summarized by the inventor when designing the dental treatment robot.

As summarized in FIG. 1, the size of the common surgical robot is quite large since it covers a large surgical area, whereas the size of the dental treatment robot can be relatively very small since it covers a small treatment area of about 10 to 15 mm. Next, since the common surgical robot operates on a patient's soft tissue, it is difficult to implement the fixing between the patient's body and the robot. The patient breathes during surgery even under general anesthesia so that his/her chest moves, which causes the body to move even a little. For this reason, it is very difficult to use a fixed reference point. On the other hand, in the case of the dental treatment robot, a treatment target tooth consists of very hard tissue. When the treatment target tooth is firmly fixed, the robot can be controlled very simply. Further, the common surgical robot can significantly affect the patient's life when malfunctioning, whereas the dental treatment robot is not employed for performing a procedure that affects the patient's life, so that the introduction of the dental treatment robot is less burdensome to dentists.

Among the characteristics of the dental treatment robot summarized in FIG. 1, the inventor has noted that the position of the robot may be relatively fixed with respect to the tooth.

As described above, the conventional surgical robot cannot completely control the movement of the patient. The tooth moves when the patient moves. For example, assuming that a preparation procedure is performed in a cross shape on the treatment target tooth, a sensor for measuring the patient's movement is required according to the prior art, and it is necessary to sense how much the patient moves through the sensor and perform correction. However, if the tooth subjected to the preparation procedure is relatively fixed with respect to the robot, the sensor for measuring the patient's movement as above is not required. Further, no correction operation is required, and errors due to the sensing and the correction operation can be eliminated in advance. If the movement of the treatment target tooth is controlled in this way when the robot treatment is performed, the preparation procedure can be performed easily and accurately just as an industrial milling machine performs machining on a workpiece.

The inventor has found that when a robot treatment is performed on a tooth of a patient, the fixing of the tooth is almost completely guaranteed by fixing the tooth in a state in which the patient's body is fixed as the patient is seated and the patient's head and jaw are fixed in advance, rather than fixing only the tooth, which led to the conception of the invention. Here, the expression “completely” does not mean that the tooth can never be moved. As described above, the treatment area of the dental treatment robot is limited to about 10 to 15 mm. When the treatment area is large, caution is required because even a small movement may cause an error to expand beyond an allowable range. However, in the case of tooth preparation and cavity filling procedures, the degree of fixing as described above exhibits quite satisfactory fixing performance within the limited treatment area.

FIG. 2 illustrates a procedure of fixing a patient's body when tooth preparation is carried out with a dental treatment robot according to one embodiment of the invention. Here, the patient's body, head, jaw, and tooth are all fixed to an immobile base of the dental treatment robot according to the invention.

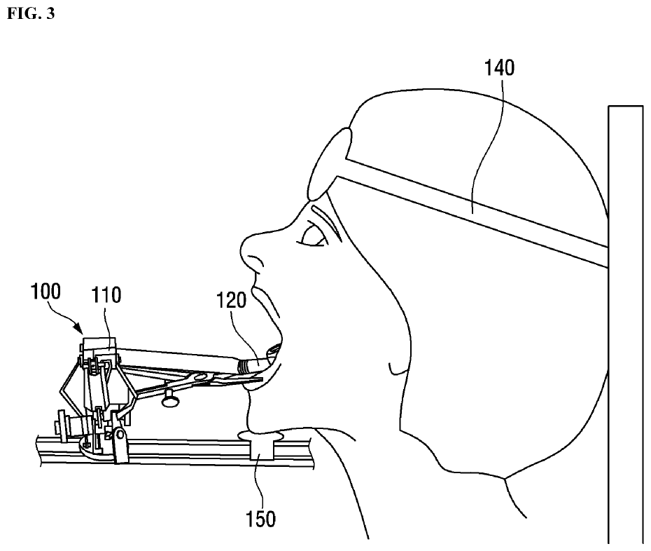

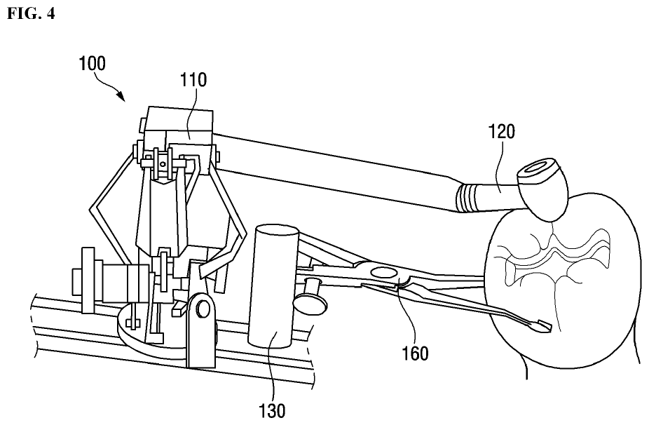

FIG. 3 illustrates a situation of performing a tooth preparation procedure with the dental treatment robot in a state in which the patient's body, head, jaw, and tooth are fixed, and FIG. 4 specifically illustrates the dental treatment robot and its tooth fixing unit according to one embodiment of the invention.

Referring to FIGS. 3 and 4, a dental treatment robot 100 according to one embodiment of the invention comprises a robot arm 110, a handpiece 120, and a base 130. The robot 100 may further comprise a head fixing unit 140, a jaw fixing unit 150, and a tooth fixing unit 160.

As conceptually shown in FIG. 3, the patient's body, head, and jaw are fixed through the head fixing unit 140, the jaw fixing unit 150, and the like in a state in which the patient is seated in a chair. Although not shown in FIG. 3, a fixing device may be additionally installed to ear canals of the patient. In this case, rotation of the patient's head is suppressed so that the patient's head may be more securely fixed. In a state in which the patient's body, head, and jaw are fixed, a separate mouth gag may be used to keep the patient's mouth open.

As shown in FIG. 3, the handpiece 120 is attached to the robot arm 110, and the length of the robot arm 110 may be configured as short as possible to reduce errors caused by the movement of the robot arm 110, which is advantageous for improving precision. The use of the mouth gag may advantageously affect the fixing of the jaw. When the patient's lips interfere with the movement of the handpiece 120, a lip retractor may be used. In this case, protection of the lip region is additionally achieved.

Since minute movements may occur even if the patient's head and jaw are fixed, it is necessary to firmly fix the dental treatment robot to the hard tissue of the tooth.

To this end, the treatment target tooth or a tooth around the treatment target tooth is fixedly coupled to the tooth fixing unit 160 that is fixedly connected to the base 130 of the robot. It can be seen in FIG. 4 that a tooth gripper is firmly coupled to the tooth in a state in which the tooth gripper is fixedly connected to the base 130 of the robot. The tooth fixing unit 160 that is fixed to the base 130 of the robot and fixedly coupled to the tooth may be a gripper as shown in FIG. 4. Alternatively, the tooth fixing unit 160 may be a clamp, and may be implemented with any fixing device available at the time of filing of the present application, as long as it can firmly fix the tooth and can be fixed to the base 130 of the robot.

Dental silicone may be additionally applied to the tooth fixing area so that the tooth is more firmly fixed. The robot arm 110 shown in FIG. 4 may be implemented with any robot arm as long as it is basically capable of moving in directions of three axes (x, y, z) (not excluding the robot arm capable of moving in directions of four or more axes). When the three-axis robot arm is used as above, it is preferable to fix the patient such that the arrangement of an occlusal plane is appropriate for the movement of the robot arm. When the arrangement is not achieved by just fixing the patient, the dental treatment robot 100 may be provided with a measurement device (e.g., an occlusal plane measurement device) that provides a measurement result for correcting the posture of the robot arm 110, and the measurement result may lead to a plane tilt correction.

As will be described below, the robot arm 110 of the dental treatment robot 100 according to the invention causes the handpiece 120 to repeatedly move in a straight line defined by two inputted endpoints. Here, when the robot arm 110 is linearly moved in a state in which the z-axis of the robot arm 110 is rotationally aligned to be parallel to a longitudinal axis of the tooth on the basis of the measurement result of the measurement device, the handpiece 120 connected to the robot arm 110 may carry out tooth preparation while repeatedly moving at the same depth. In order to increase the depth of the tooth preparation step by step, a simple control may be performed to move the robot arm 110 stepwise in the z-axis direction.

A laser device known in the art at the time of filing of the present application may be employed for the same purpose as the above-described occlusal plane measurement device. A method of adjusting the occlusal plane of the patient and the base 130 of the robot to be in parallel using the laser device will be briefly described. According to this method, the patient is seated first and then the patient's head is fixed to a headrest. A hair tying method using a strap may be used as the fixing method. Next, the patient's mouth is opened and fixed with a mouth gag, and the patient's jaw is placed. The rotation of the head may be minimized by placing a fixing device in ear canals of the patient. The occlusal plane of the patient and the base 130 of the robot are adjusted to be in parallel. To this end, laser light is irradiated to the patient's face so that alignment with the occlusal plane may be easily achieved.

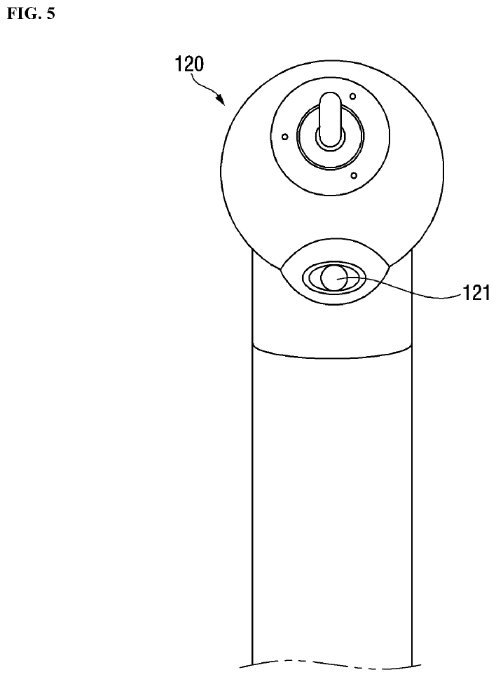

FIG. 5 illustrates a handpiece to which a camera is attached. A dental handpiece 120 is used for tooth preparation. The dental handpiece 120 includes a high-speed handpiece using an air turbine (operated at 300,000 to 400,000 RPM) and a low-speed handpiece using an electric motor.

The handpiece 120 applied to the dental treatment robot 100 according to one embodiment of the invention preferably includes a camera 121 and an LED light (not shown) around the head. An image of the treatment area captured through the camera 121 installed on the handpiece 120 shown in FIG. 5 may be outputted to a monitor. A dentist may check decay in the tooth shown on the monitor, and may determine a start point and a section of the movement of the handpiece for tooth preparation. The depth of the decay removed each time may be 0.1 mm to 0.5 mm, and the dentist may check the remaining decay and continue the tooth preparation.

The handpiece 120 is firmly connected to the robot arm 110, and the connection of the handpiece 120 to the robot arm 110 is preferably formed in a detachable manner. The detachable connection may be advantageously utilized when injecting a resin after the tooth preparation procedure is completed. If a single robot arm is employed, the handpiece 120 for the tooth preparation is removed from the robot arm 110 and a tool for the resin injection may be coupled in place of the handpiece 120 when the tooth preparation is completed and then the resin injection procedure is performed. If two or more robot arms are employed, the handpiece 120 for the tooth preparation and the tool for the resin injection may be mounted on separate robot arms, respectively. In this case, the robot arm 110 to which the handpiece 120 for the tooth preparation is attached is retracted from the treatment area when the tooth preparation procedure is completed, and another robot arm to which the tool for the resin injection is attached may be brought to the treatment area.

Meanwhile, the handpiece 120 sprays air and water together. This serves to protect the dental nerves (or dental pulp) by cooling the tooth during the preparation procedure. However, the water sprayed from the handpiece 120 adheres to a cover glass for protecting the lens of the camera 121 and interferes with the image capture through the camera. In this regard, a solution thereto is required.

The inventor proposes the following two methods as the solution to the above problem.

The first method is to remove water droplets by spraying air that does not contain water toward the cover of the lens.

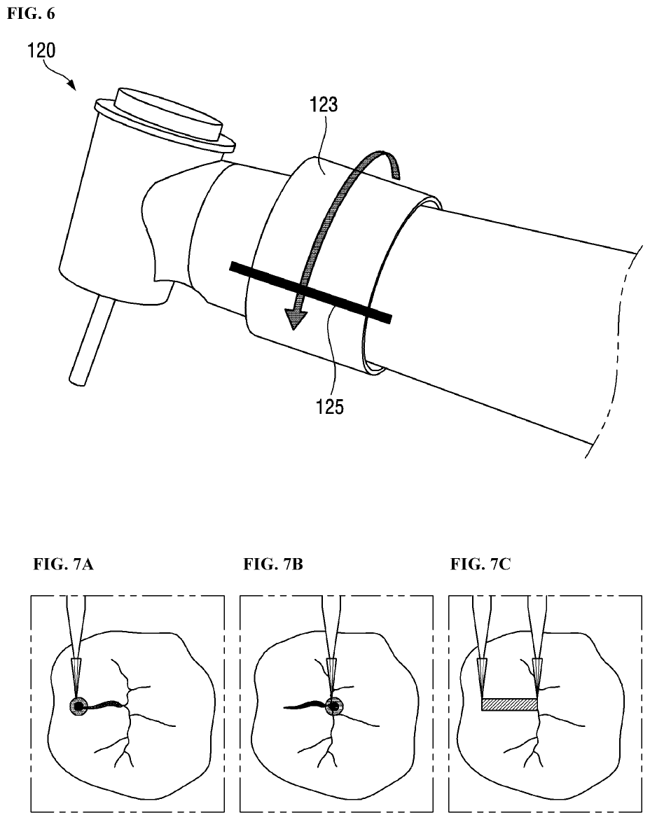

The second method is to design the lens cover to be rotatable and install a wiper structure to traverse the lens cover. FIG. 6 shows such a wiper structure. A component indicated by a black straight line in FIG. 6 is a wiper. In the structure shown in FIG. 6, when a lens cover 123 is rotated in a state in which water droplets adhere thereto, a wiper 125 may effectively remove the water droplets adhering to the lens cover 123.

FIGS. 7A-7C illustrate a situation of performing a tooth preparation procedure with a dental treatment robot according to one embodiment of the invention. First, a dentist identifies tooth decay from an image transmitted from an intraoral camera and displayed on a monitor. Next, the dentist places a bur of a handpiece for dental treatment near the tooth decay while looking at the screen. Next, a robot arm is moved downward in the z-axis direction so that the bur contacts the tooth in a state in which the bur is not rotated. Next, the dentist clicks on the contact point of the bur on the screen while the bur and the tooth are in contact. At this time, zero adjustment is made. Next, the dentist clicks on both ends of a line for which the tooth preparation is necessary, and inputs a tooth preparation depth on the z-axis. FIGS. 7A-7C show this step. It can be seen from FIGS. 7A-7C that the bur of the handpiece for the tooth preparation rotates and moves from one endpoint of the line inputted by the dentist to the other endpoint to remove the tooth decay.

As an alternative embodiment, it is also possible to carry out the tooth preparation while adjusting the position of the handpiece in real time using a joystick-type device. As described above, when using the dental treatment robot according to the invention, not only the patient's body, head, and jaw are fixed but also the treatment target tooth (or a tooth around the treatment target tooth) is fixedly connected to the base (which is not subjected to the movement of the dental treatment robot) by the fixing device, so that there is no or negligible movement of the treatment target tooth during the treatment. Therefore, as shown in FIGS. 7A-7C, the tooth decay may be effectively removed by the linear preparation carried out by repeated movements between the start and end points.

When the tooth is moved, the degree of the tooth movement should be sensed first, and the section of the movement for the tooth decay removal will not be as simple as a straight line but will be formed as a line of a complex path in a three-dimensional space. It is difficult to control a handpiece having a bur rotating at a high speed along such a path, and soft tissue in the patient's mouth may be injured by the high-speed rotating bur if the control fails by any chance.

That is, the distinct features of the dental treatment robot according to the invention compared to the conventional robot are (i) fixing a treatment target tooth or a tooth around the treatment target tooth to the robot device in addition to fixing a patient's body, head, and jaw, and (ii) carrying out tooth preparation by linear movement of the robot arm while the movement in the z-axis direction is fixed. The movement of the robot arm in the z-axis direction, i.e., the tooth preparation in the depth direction may be performed stepwise.

Restorative treatment for filling a cavity is necessary after the tooth preparation is completed. The most frequently performed tooth decay treatment is resin treatment. By filling the cavity with a resin, healthy food intake may be ensured to the patient. An injection device capable of injecting a flowable resin may be connected to the robot arm. The dentist may position a nozzle of the injection device at the cavity while looking at the monitor, and operate the resin injection device to inject the resin into the cavity. Thereafter, when irradiation of light for curing the resin is performed, the resin is cured and the treatment is completed. The area where the resin is injected is the same as the area of the tooth preparation. Accordingly, the movement area of the resin injection device may be displayed on the monitor in the same manner as described above, and the resin injection may be accurately performed by the movement of the robot arm to which the resin injection device is attached in the same manner as above.

Although the present invention has been described above in terms of specific items such as detailed elements as well as the limited embodiments and the drawings, they are only provided to help more general understanding of the invention, and the present invention is not limited to the above embodiments. It will be appreciated by those skilled in the art to which the present invention pertains that various modifications and changes may be made from the above description.

Therefore, the spirit of the present invention shall not be limited to the above-described embodiments, and the entire scope of the appended claims and their equivalents will fall within the scope and spirit of the invention.

What is claimed is:

1. A dental treatment robot, comprising:
a head fixing unit configured to fix a head of a patient;
a jaw fixing unit configured to fix a jaw of the patient in an opened state;
a tooth fixing unit configured to fix a treatment target tooth or a tooth around the treatment target tooth;
a robot arm capable of moving in directions of at least three axes;
a handpiece that is detachably attached to the robot arm and includes a rotating part for tooth preparation; and
a base whose movement is fixed,
wherein the head fixing unit, the jaw fixing unit, and the tooth fixing unit are connected and fixed to the base, and the robot arm moves on the basis of the base to carry out the tooth preparation.

2. The dental treatment robot of claim 1, wherein the robot arm repeatedly moves in a straight line defined by two inputted endpoints to remove tooth decay,
wherein the robot arm repeatedly moves in the straight line in a state in which a direction of a longitudinal axis of the tooth is aligned parallel to a z-axis, and
wherein the robot arm moves stepwise in the z-axis direction.

3. The dental treatment robot of claim 2, wherein the handpiece includes a camera, and the camera is configured to transmit an image of a treatment area to a monitor.

4. The dental treatment robot of claim 3, wherein the monitor is a touch screen monitor, and the two endpoints of the straight line are inputted by touches on the monitor.

5. The dental treatment robot of claim 1, further comprising an injection device configured to inject a resin for filling a cavity, wherein the injection device is detachably attached to the robot arm.

6. The dental treatment robot of claim 3, wherein the handpiece includes a lens cover for protecting a lens of the camera,
wherein the lens cover is capable of rotating in a state in which the lens cover is mounted on the handpiece, and
wherein a wiper is installed to traverse the lens cover, so that foreign substances adhering to the lens cover are removed by the wiper when the lens cover is rotated.
